(19) United States
(12) Patent Application Publication
(10) Pub. No.: US 2025/0312575 A1
(43) Pub. Date: Oct. 9, 2025

(54) Localized Intra-Arterial Drug Delivery Device

(71) Applicant: BOARD OF TRUSTEES OF THE UNIVERSITY OF ARKANSAS, Little Rock, AR (US)

(72) Inventors: Sam Stephens, Fayetteville, AR (US); Sumith Ranil Wickramasinghe, Fayetteville, AR (US); Max Green, Fayetteville, AR (US); Morten Jensen, Fayetteville, AR (US); Kaitlyn Elmer, Fayetteville, AR (US)

(73) Assignee: BOARD OF TRUSTEES OF THE UNIVERSITY OF ARKANSAS, Little Rock, AR (US)

(21) Appl. No.: 19/173,203

(22) Filed: Apr. 8, 2025

Related U.S. Application Data
(60) Provisional application No. 63/631,180, filed on Apr. 8, 2024.

(51) Int. Cl. A61M 25/10; A61M 1/00

(52) U.S. Cl. CPC A61M 25/10; A61M 1/85; A61M 2025/105; A61M 2025/1052; A61M 2025/109

(57) ABSTRACT

The present disclosure pertains to an active agent delivery device for insertion into a blood vessel that includes: (1) a proximal end, a distal end, and an exterior surface between the proximal and distal ends; (2) a lumen extending from the proximal end to the distal end beneath the exterior surface and operable to facilitate blood flow through the blood vessel; (3) an active agent dispenser associated with the exterior surface and operable to release an active agent into the blood vessel; (4) an irrigation channel operable to remove materials from the blood vessel; and (5) an expandable structure at or near the distal end and operable to expand downstream the blood vessel region containing the device and localize released actives agent proximal to the region containing the device. The present disclosure also pertains to methods of using the device to deliver active agents into blood vessels of subjects.

### CROSS-REFERENCE TO RELATED APPLICATIONS

This application claims priority to U.S. Provisional Patent Application No. 63/631,180, filed on Apr. 8, 2024. The entirety of the aforementioned application is incorporated herein by reference.

### BACKGROUND

A need exists for more effective devices and methods for treating blocked blood vessels. Numerous embodiments of the present disclosure aim to address the aforementioned need.

### SUMMARY

In some embodiments, the present disclosure pertains to an active agent delivery device that is operable for insertion into a blood vessel. In some embodiments, the active agent delivery device includes: (1) a proximal end, a distal end, and an exterior surface between the proximal end and the distal end; (2) a lumen extending from the proximal end to the distal end beneath the exterior surface, where the lumen is operable to facilitate blood flow through the blood vessel; (3) an active agent dispenser associated with the exterior surface, where the active agent dispenser is operable to release at least one active agent into the blood vessel; (4) an irrigation channel operable to remove materials from the blood vessel; and (5) an expandable structure at or near the distal end, where the expandable structure is operable to expand downstream the blood vessel region containing the active agent delivery device and thereby localize the released active agent proximal to the blood vessel region containing the device.

Additional embodiments of the present disclosure pertain to methods of delivering an active agent into a blood vessel of a subject by utilizing the active agent delivery devices of the present disclosure. In some embodiments, such methods include: (1) inserting an active agent delivery device into the blood vessel; (2) expanding the expandable structure downstream the blood vessel region containing the active agent delivery device; and (3) delivering at least one active agent from the active agent dispenser into the blood vessel. During operation, the lumen of the active agent delivery device may facilitate blood flow through the blood vessel, the irrigation channel may remove materials from the blood vessel, and the expanded expandable structure may localize the released active agents proximal to the blood vessel region containing the active agent delivery device.

### DRAWINGS

A better understanding of the present disclosure can be obtained when the following detailed description is considered in conjunction with the following drawings, in which:

FIG. 1 illustrates an embodiment of an active agent delivery device 10 in accordance with various aspects of the present disclosure.

FIGS. 2A-2C provide different positions of a valve stopper 24 of active agent delivery device 10 shown in FIG. 1, including a deployment position where the valve stopper 24 blocks both the bypass lumen 22 and entry site of the active agent dispenser 14 (FIG. 2A), an inflation position where the valve stopper 24 allows fluids (e.g., saline) to enter bypass lumen 22 and reach expandable structure 16 while still blocking the entry site of active agent dispenser 14 (FIG. 2B), and a dispensing position, where valve stopper 24 blocks the distal end of the bypass lumen 22, thus keeping the expandable structure 16 inflated (FIG. 2C).

FIGS. 3A1-3B2 provide various depictions of active agent dispenser 14 of active agent delivery device 10 shown in FIG. 1, including at a tilted position inside a clot (FIGS. 3A1-3A2), and an un-tilted position inside the clot (FIGS. 3B1-3B2).

FIGS. 4A-4H illustrate a process of utilizing the active agent delivery device 10 shown in FIG. 1 for delivering an active agent into a blood vessel.

FIG. 5 depicts a sliced view of an alternative active agent delivery device. The net, the active agent dispenser inlet and outlet, and the lumen are labelled.

FIGS. 6A-6C illustrate a process of utilizing the active agent delivery device shown in FIG. 5 for delivering an active agent into a blood vessel. FIG. 6A shows a blood clot in the blood vessel. FIG. 6B shows the active agent delivery device entering the blood clot, with a net deployed, and with active agents (i.e., thrombolytic agents) being released. FIG. 6C shows the net catching clot fragments as the clot begins to break up while the area continues to be flooded with thrombolytic agents.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. If one or more of the incorporated literature and similar materials defines a term in a manner that contradicts the definition of that term in this application, this application controls.

Numerous conditions are associated with blocked blood vessels. For instance, ischemic stroke is a life-threatening condition often characterized by either a thrombus or emboli formation in an artery in the brain. Treatments seek to quickly restore blood flow, typically using mechanical thrombectomy or intravenous thrombolysis methods.

Mechanical thrombectomy uses various mechanisms to pull the bulk of the clot out of the artery. Such a method quickly restores blood vessel patency. Additionally, such a method provides a tool for patients that cannot be treated with intravenous tissue plasminogen activators (tPAs) due to prolonged lapses from the patient's last seen normal condition.

Nonetheless, methods of treating blocked blood vessels have numerous limitations. For instance, the drawbacks of mechanical thrombectomy are a tendency for small clot pieces to break off and cause further blockages downstream. Intravenous thrombolysis uses clot dissolving agents to dissolve the clot and restore blood flow. However, potential issues with thrombolysis approaches include, without limitation, increased time before blood flow is restored, occlusion downstream of an initial clot, and symptomatic intracranial hemorrhage.

As such, a need exists for more effective devices and methods for treating blocked blood vessels. Numerous embodiments of the present disclosure aim to address the aforementioned need.

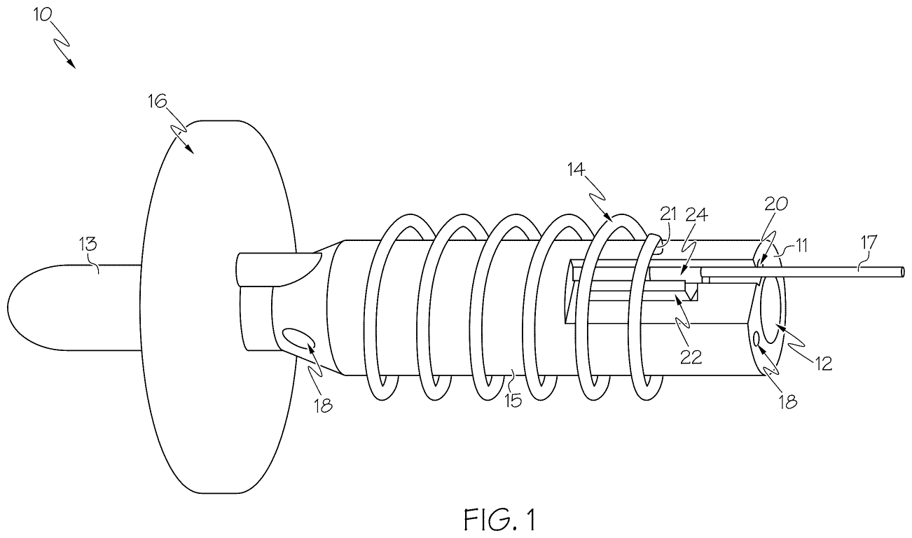

In some embodiments, the present disclosure pertains to an active agent delivery device that is operable for insertion into a blood vessel. With reference to FIGS. 1, 2A-2C, and 3A1-3B2 for illustrative purposes, active agent delivery device 10 generally includes a proximal end 11, a distal end 13, and an exterior surface 15 between the proximal end 11 and the distal end 13. Additionally, active agent delivery device 10 includes a lumen 12 extending from the proximal end 11 to the distal end 13 beneath the exterior surface 15. Lumen 12 is generally operable to facilitate blood flow through a blood vessel that contains an active agent delivery device of the present disclosure.

In some embodiments, active agent delivery device 10 also includes an active agent dispenser 14 associated with the exterior surface 15. Active agent dispenser 14 is generally operable to release at least one active agent into a blood vessel that contains an active agent delivery device of the present disclosure.

In some embodiments, active agent delivery device 10 also includes an irrigation channel 18 that is operable to remove materials from a blood vessel that contains the device. In some embodiments, active agent delivery device 10 also includes an expandable structure 16 at or near the distal end 13 that is operable to expand downstream a blood vessel region that contains the device. In some embodiments, expandable structure 16 is operable to localize released active agents from active agent dispenser 14 proximal to the blood vessel region containing the device.

Additional embodiments of the present disclosure pertain to methods of delivering an active agent into a blood vessel of a subject. In some embodiments, such methods generally include inserting an active agent delivery device 10 into the blood vessel and delivering at least one active agent from the active agent dispenser 14 into the blood vessel. In some embodiments where active agent delivery device 10 includes an expandable structure 16, the methods of the present disclosure may also include a step of expanding the expandable structure 16 downstream the blood vessel region containing the device. In some embodiments, the expansion occurs prior to the delivery of the active agent in order to localize released active agents proximal to the blood vessel region containing the device. In some embodiments where active agent delivery device 10 includes an irrigation channel 18, the irrigation channel 18 may be utilized to remove materials from the blood vessel.

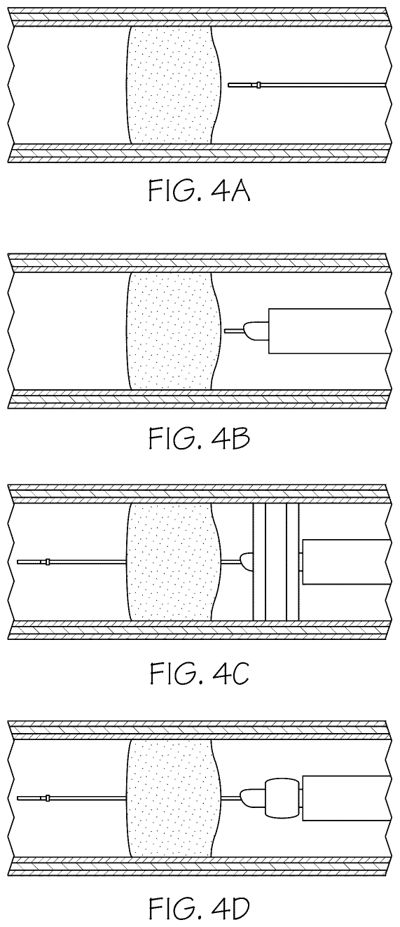

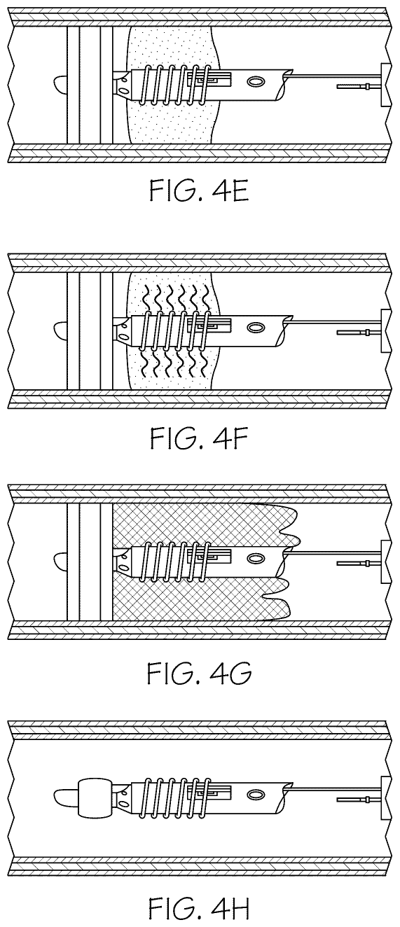

The methods of the present disclosure can operate in various modes. For instance, in some embodiments illustrated in FIGS. 4A-4H, a guidewire may first be inserted into a blood vessel to locate a blocked area of the blood vessel (FIG. 4A). Thereafter, active agent delivery device 10 may be inserted into the blood vessel (FIG. 4B). Expandable structure 16 may then be expanded to center the guidewire at the blocked blood vessel (FIG. 4C). Expandable structure 16 may then be deflated while the active agent delivery device is inserted into the blocked blood vessel (FIG. 4D). Expandable structure 16 may then be expanded (FIG. 4E) while active agents are delivered from active agent dispenser 14 to the blood vessel (FIG. 4F). During this process, the expanded expandable structure 16 may be utilized to localize the released active agents from active agent dispenser 14 proximal to the blood vessel region containing the device, thereby resulting in a more effective treatment of the blocked blood vessel (FIGS. 4E-4G). During this process, irrigation channel 18 may be utilized to remove materials from the blood vessel, such as released active agents, dissolved clots, and/or debris (FIG. 4G). Thereafter, the expanded expandable structure 16 may be deflated and the active agent delivery device 10 may be removed from the blood vessel (FIG. 4H).

As set forth in more detail herein, the active agent delivery devices of the present disclosure can have various structures and arrangements. Additionally, the methods of the present disclosure may be utilized to deliver various active agents to various blood vessels for various purposes.

### Lumens

Lumens generally refer to openings or cavities of active agent delivery devices of the present disclosure that are operable to facilitate blood flow through a blood vessel that contains the device. The active agent delivery devices of the present disclosure can include various types of lumens. For instance, with reference to lumen 12 in FIG. 1 for illustrative purposes, lumen 12 may be located centrally within active agent delivery device 10. In some embodiments, lumen 12 may include an inlet region at or near proximal end 11, and an outlet region at or near distal end 13. In some embodiments, the inlet region and the outlet region are operable to facilitate blood flow through a blood vessel that contains the active agent delivery device.

### Active Agent Dispensers

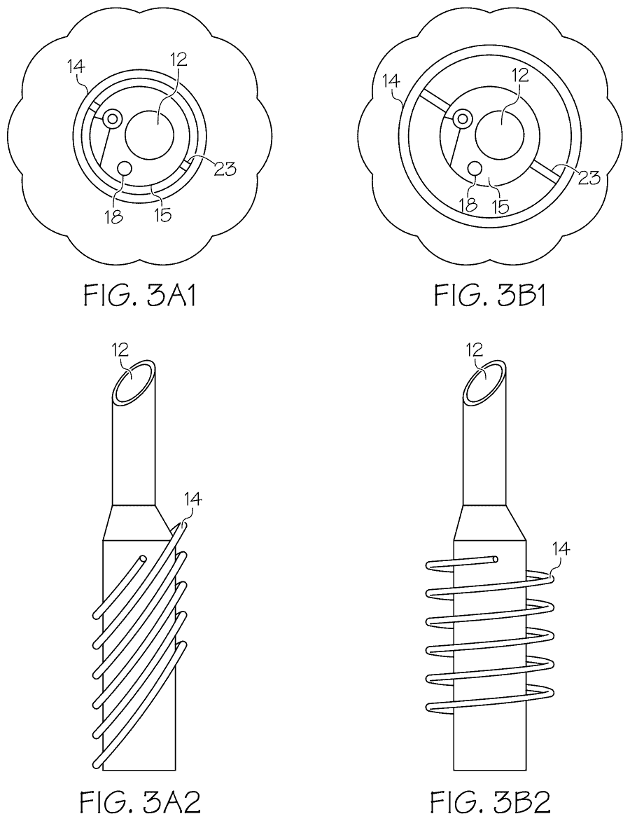

Active agent dispensers generally refer to structures that are operable to release at least one active agent into a blood vessel that contains an active agent delivery device of the present disclosure. The active agent delivery devices of the present disclosure can include various types of active agent dispensers. For instance, with reference to active agent dispenser 14 in FIGS. 1 and 3A1-3B2 for illustrative purposes, active agent dispenser 14 may be in the form of a helical membrane wound around exterior surface 15 of active agent delivery device 10. In some embodiments, active agent dispenser 14 is spiral shaped. In some embodiments, active agent dispenser 14 is porous.

In some embodiments, an active agent dispenser of the present disclosure (e.g., active agent dispenser 14) is a permeable, hollow fiber that allows an active agent (e.g., a thrombolytic agent) to be placed in direct contact with a clot in a blood vessel. In some embodiments, a helical shape of an active agent dispenser (e.g., active agent dispenser 14) provides a high surface area for active agent delivery and allows the active agent dispenser to remain in contact with a blood vessel for direct contact with an active agent (e.g., a thrombolytic agent).

In some embodiments illustrated in FIGS. 1 and 3A1-3B2, active agent dispenser 14 may be operable to expand outward radially to follow a surface of a blood vessel. In some embodiments, active agent dispenser 14 is operable to expand outward radially by switching from a tilted position (e.g., FIGS. 3A1-3A2) to an un-tilted position (e.g., FIGS. 3B1-3B2). In some embodiments further illustrated in FIGS. 3A1-3B2, such an expansion is achieved with the help of a wire 23 (e.g., a nitinol wire) that is attached to active agent dispenser 14 and exterior surface 15.

### Irrigation Channels

Irrigation channels generally refer to channels that are operable to remove materials from a blood vessel that contains an active agent delivery device of the present disclosure. The active agent delivery devices of the present disclosure can include various types of irrigation channels. For instance, with reference to irrigation channel 18 in FIG. 1 for illustrative purposes, irrigation channel 18 may be in the form of a channel that is positioned within active agent delivery device 10. In some embodiments, irrigation channel 10 extends from proximal end 11 to the distal end 13 in parallel with lumen 12. In some embodiments, irrigation channel 18 is positioned at distal end 13 of active agent delivery device 10.

The irrigation channels of the present disclosure may have various functions. For instance, in some embodiments, the irrigation channels of the present disclosure are operable to remove released active agents, dissolved clots, and/or debris from a blood vessel. In some embodiments, an irrigation channel of the present disclosure may draw an active agent out of a blood vessel after a clot has been sufficiently dissolved, thereby preventing it from mixing with the rest of the circulatory system.

In some embodiments, the irrigation channels of the present disclosure may also be operable to interface with a suction device for the removal of materials from a blood vessel. For instance, in some embodiments, the irrigation channels of the present disclosure are operable to interface with an external device that provides suction for such removal. In some embodiments, the suction device may also monitor the volume of active agent removed. In some embodiments, the methods of the present disclosure may also include a step of coupling an irrigation channel with a suction device such that the suction device facilitates the removal of materials from the blood vessel.

In some embodiments, an active agent dispenser of the present disclosure may diffuse a fluid (e.g., saline) to facilitate filtration and reabsorption through an irrigation channel of the present disclosure. In some embodiments, the fluid (e.g., saline) may be used to assist in aspiration at a proximal end of an active agent delivery device to push an active agent into inlets of the device.

### Expandable Structures

Expandable structures generally refer to structures that are operable to expand downstream a blood vessel region that contains the active agent delivery device of the present disclosure. In some embodiments, the expandable structures of the present disclosure may also be operable to localize released active agents proximal to a blood vessel region containing an active agent delivery device of the present disclosure. In some embodiments, such localization may allow for higher active agent concentration at the site of the blood vessel than if the active agent were administered systemically. In some embodiments, such localization may also permit different, potentially more potent active agents to be used than if the active agent was allowed to travel systemically throughout the body.

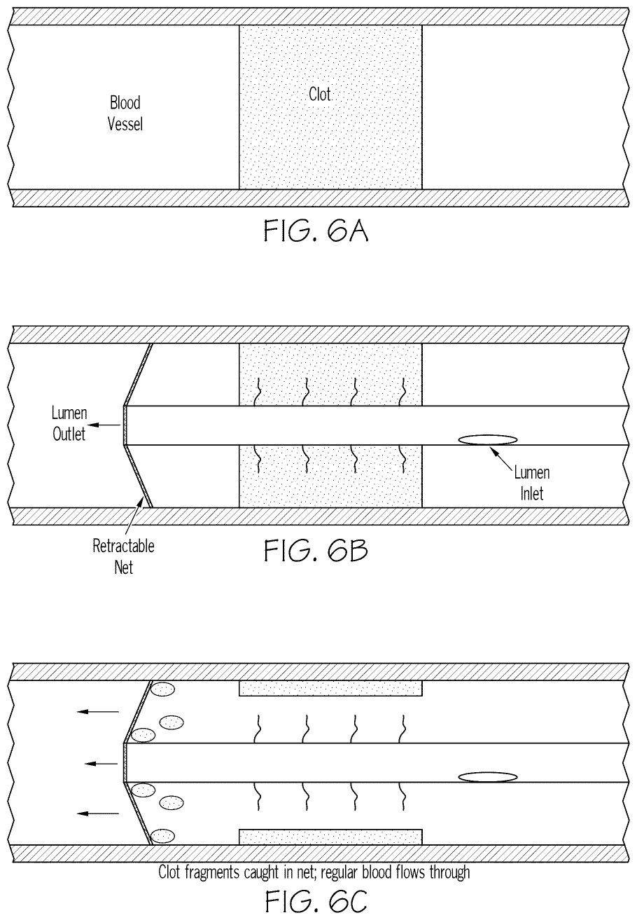

The active agent delivery devices of the present disclosure can include various types of expandable structures. For instance, in some embodiments, an expandable structure may include a balloon, a net, or combinations thereof. In some embodiments, the expandable structure includes a balloon, such as balloon 16 shown in FIG. 1. In some embodiments, the expandable structure includes a net, such a retractable net shown in FIGS. 6B-6C.

In some embodiments, the expandable structures of the present disclosure may be made by heated pressurization to fit a device-specific mold. In some embodiments, the expandable structures of the present disclosure can also be used to aid in centering a guide wire before penetrating a blood vessel (e.g., FIG. 4C). In some embodiments, this ensures that an active agent delivery device is implemented in a more central location of a blood vessel rather than towards the outer edges of the blood vessel.

### Inflating Channels

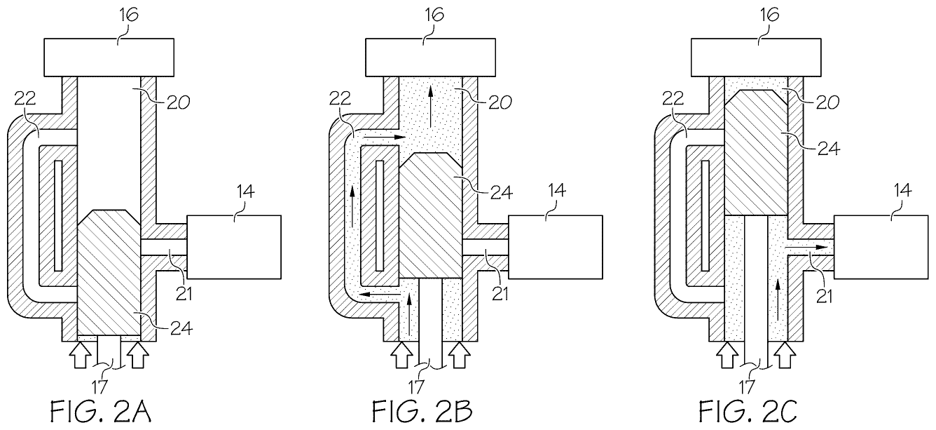

In some embodiments illustrated in FIGS. 1 and 2A-2C, the active agent delivery devices of the present disclosure also include an inflating channel 20 that is associated with expandable structure 16. Inflating channel 20 is generally operable to expand expandable structure 16 through the flow of air or fluids into expandable structure 16. In some embodiments, inflating channel 20 is also operable to interface with a fluid or air delivery device for expansion of expandable structure 16. In some embodiments, inflating channel 20 extends from proximal end 11 to distal end 13 of inflating channel 20.

In some embodiments where the active agent delivery device further includes an inflating channel 20 associated with expandable structure 16, the methods of the present disclosure may also include a step of expanding expandable structure 16 by flowing air or fluids into inflating channel 20. In some of such embodiments, the methods of the present disclosure also include a step of interfacing inflating channel 20 with a fluid or air delivery device for expansion of expandable structure 16.

### Active Agent Delivery Channels

In some embodiments illustrated in FIGS. 1 and 2A-2C, the active agent delivery devices of the present disclosure also include an active agent delivery channel 21 that is associated with active agent dispenser 14. Active agent delivery channel 21 is generally operable to deliver one or more active agents to active agent dispenser 14. In some embodiments, active agent delivery channel 21 is also operable to interface with an active agent delivery pump for active agent delivery to active agent dispenser 14.

In some embodiments where the active agent delivery device further includes active agent delivery channel 21 associated with active agent dispenser 14, the step of active agent delivery to a blood vessel includes delivering one or more active agents to active agent dispenser 14 through active agent delivery channel 21. In some of such embodiments, the methods of the present disclosure also include a step of interfacing active agent delivery channel 21 with an active agent delivery pump for active agent delivery to active agent dispenser 14.

### Interconnections Between Inflating and Active Agent Delivery Channels

In some embodiments illustrated in FIGS. 1 and 2A-2C, the active agent delivery devices of the present disclosure include an inflating channel 20 and an active agent delivery channel 21 that are interconnected with one another. For instance, in some embodiments, active agent delivery device 10 includes: (1) an inflating channel 20 associated with expandable structure 16 and operable to expand expandable structure 16 through the flow of air or fluids into the expandable structure 16; (2) an active agent delivery channel 21 associated with active agent dispenser 14 and inflating channel 20, where the active agent delivery channel 21 is in air or fluid communication with active agent dispenser 14 and operable to deliver one or more active agents to active agent dispenser 14; and (3) a valve stopper 24 associated with inflating channel 20 and active agent delivery channel 21 and operable to control the flow of fluids or air to inflating channel 20 and active agent delivery channel 21.

In some embodiments, active agent delivery device 10 also includes a bypass lumen 22 connected to a first end and a second end of inflating channel 20. In some embodiments, bypass lumen 22 has both ends connected to inflating channel 20 (e.g., a first end and a second end of the inflating channel 20). In some embodiments, the bypass lumen 22 may allow for fluid to bypass the connection between the active agent dispenser 14 and the inflating channel 20 so that fluid can expand the expandable structure 16.

In some embodiments, valve stopper 24 is operable to cease air and fluid flow to inflating channel 20 and active agent delivery channel 21 (e.g., FIG. 2A), direct air and fluid flow to inflating channel 20 but not active agent delivery channel 21 (e.g., FIG. 2B), direct air and fluid flow to active agent delivery channel 21 but not inflating channel 20 (e.g., FIG. 2C), or direct air and fluid flow to active agent delivery channel 21 and inflating channel 20. In some of such embodiments, the methods of the present disclosure include one or more of the following steps: positioning valve stopper 24 in a first position to cease air or fluid flow to inflating channel 20 and active agent delivery channel 21 (e.g., FIG. 2A), positioning valve stopper 24 in a second position to direct air or fluid flow to inflating channel 20 but not active agent delivery channel 21 (e.g., FIG. 2B), positioning valve stopper 24 in a third position to direct air or fluid flow to active agent delivery channel 21 but not inflating channel 20 (e.g., FIG. 2C), and/or positioning the valve stopper in a fourth position to direct air or fluid flow to active agent delivery channel 21 and inflating channel 20.

In some embodiments, the methods of the present disclosure include the following steps: (1) positioning the valve stopper 24 in a first position to direct air or fluid flow to the inflating channel 20 but not the active agent delivery channel 21, where the air or fluid flow expands the expandable structure 16 downstream a blood vessel region containing active agent delivery device 10 (e.g., FIG. 2B); and (2) positioning the valve stopper 24 in a second position to direct air or fluid flow to the active agent delivery channel 21 but not the inflating channel 20, where the air or fluid flow delivers at least one active agent from the active agent dispenser 14 into a blood vessel while the valve stopper maintains the expandable structure 16 in an expanded position (e.g., FIG. 2A).

In a deployment position shown in FIG. 2A, valve stopper 24 may block both the bypass lumen 22 and active agent delivery channel 21. This prevents the expandable structure 16 from expanding and active agents and/or fluids (e.g., saline) from entering the active agent dispenser 14. In an inflation position shown in FIG. 2B, valve stopper 24 may allow air or fluids (e.g., saline) to enter bypass lumen 22 and reach the expandable structure 16 while still blocking active agent delivery channel 21. In a dispensing position shown in FIG. 2C, valve stopper 24 may block the distal end of the bypass lumen 22, thus keeping the expandable structure 16 expanded. In this position, the stopper valve 24 has advanced past the connection between the active agent dispenser 14 and the inflating channel 20, which allows active agents to enter active agent dispenser 14 through active agent delivery channel 21 and be delivered to a blood vessel. In some embodiments illustrated in FIGS. 1 and 2A-2C, valve stopper 24 may be actuated by a wire 17 (e.g., a stiff wire) that is positioned at the proximal end 11 of active agent delivery device 10.

### Guide Wires

In some embodiments, the active agent delivery devices of the present disclosure can also include a guide wire operable to guide an active agent delivery device through a blood vessel. As such, in some embodiments, the step of inserting an active agent delivery device of the present disclosure into a blood vessel includes a step of utilizing a guide wire to guide the active agent delivery device through the blood vessel. In some embodiments, the guide wire also helps an active agent delivery device to puncture through a blood vessel.

In some embodiments, the guide wire is at or near a distal end of an active agent delivery device. In some embodiments, the guide wire is distal to an expandable structure (e.g., expandable structure 16 in FIG. 1).

### Additional Components

The active agent delivery devices of the present disclosure can also include various additional components. For instance, in some embodiments, the active agent delivery devices of the present disclosure also include a protective sheath operable to protect the active agent delivery device upon insertion into a blood vessel. In some embodiments, the active agent delivery devices of the present disclosure also include a bushing that covers a lumen. In some embodiments, the bushing is operable to allow a lumen to function as a guidewire. In some embodiments, a bushing may keep a guidewire centered within a lumen.

### Active Agents

The active agent delivery devices and methods of the present disclosure can be utilized to deliver various active agents to blood vessels. For instance, in some embodiments, the active agents include, without limitation, blood clot dissolving drugs, thrombolytic agents, anti-tumor drugs, plasmins, or combinations thereof.

The active agents of the present disclosure can have various effects on blood vessels. For instance, in some embodiments, the released active agents may be utilized to dissolve clots in a blood vessel. In some embodiments, the released active agents may be utilized to kill tumor cells in a blood vessel.

### Blood Vessels

The active agent delivery devices and methods of the present disclosure can be utilized to deliver active agents to various blood vessels. For instance, in some embodiments, the blood vessels include, without limitation, arteries, veins, blocked blood vessels, or combinations thereof. In some embodiments, the blood vessels include blocked blood vessels. In some embodiments, the blocked blood vessels may be blocked by clots, tumors, or combinations thereof.

### Subjects

The active agent delivery devices and methods of the present disclosure can be utilized to deliver active agents to the blood vessels of various subjects. For instance, in some embodiments, the subject includes a mammal, such as a human being. In some embodiments, the subject is a human being. In some embodiments, the subject is suffering from or vulnerable to a vascular disorder. In some embodiments, the vascular disorder includes, without limitation, acute ischemic stroke (AIS), myocardial infarction, or combinations thereof.

### Applications and Advantages

The active agent delivery devices and methods of the present disclosure provide numerous advantages. For instance, in some embodiments, the devices and methods of the present disclosure can be utilized to quickly restore blood flow to a blocked blood vessel (e.g., an occluded artery) by dissolving blood clots in an expedited manner while minimizing the movement of undissolved clot particles and dissolution agents downstream. In some embodiments, such expedited restoration of blood flow in blocked blood vessels may be achieved through direct contact between active agent dispensers of a device with a blood clot, which in turn maximizes the active agent's thrombolytic effect. Additionally, the devices and methods of the present disclosure allow for increased active agent concentration near a blocked vessel and the use of alternate types of active agents because the active agent is delivered and kept locally rather than being allowed to go systemically throughout the patient's body.

As such, the devices and methods of the present disclosure can have numerous advantageous applications. For instance, in some embodiments, the methods and devices of the present disclosure may be utilized for any application in which it would be beneficial to administer an active agent locally and intra-arterially. In some embodiments, such applications can include treatment for any blood vessel occlusion by a clot, such as Acute Ischemic Stroke (AIS) and Myocardial Infarction. In some embodiments, the methods and devices of the present disclosure may be utilized in emergency medicine to treat Acute Ischemic Stroke (AIS).

In some embodiments, the methods and devices of the present disclosure may be utilized to treat tumors, such as brain tumors. The treatment of brain tumors through the use of the methods and devices of the present disclosure may be particularly advantageous because it is difficult for anti-tumor agents (e.g., chemotherapeutic agents) to cross the blood-brain barrier. As such, local delivery of anti-tumor agents through the use of the methods and devices of the present disclosure may allow a higher concentration of anti-tumor agents to be delivered to an affected site in the brain.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Design and Utilization of a Localized Intra-Arterial Drug Delivery Device

This Example illustrates the design and utilization of a localized intra-arterial drug delivery device. The development of the device began as a combined thrombectomy and thrombolysis device called the Acute Ischemic Stroke Clot Dissolver and Capture Device. The purpose of this device is to improve the current clot removal systems available to cardiovascular clinicians. The device intends to quickly restore blood flow in the occluded artery and dissolve the clot, while minimizing the movement of undissolved clot particles and dissolution agent downstream.

A purpose of the device is its application in emergency medicine to treat Acute Ischemic Stroke (AIS) (e.g., by localized delivery of a clot dissolving drug, such as plasmin), although it could be used in any situation in which local intra-arterial drug delivery is beneficial. To treat AIS, the device pierces through the clot that is causing the stroke and uses an active agent dispenser that comes into direct contact with the clot to deliver a thrombolytic drug, such as plasmin. The catheter has a central lumen which allows blood flow to be restored distal to the clot, even while the device is delivering the drug. The device deploys an expandable structure distal to the clot to keep the drug localized as well as to catch any clot fragments and prevent them from traveling downstream.

Before the device is retracted from the patient's body, aspiration through a dedicated lumen is used to remove the drug from the artery. An optional expanding net gives clinicians the ability to retrieve any potentially remaining fragments once the device is removed. Therefore, the device can provide clinicians a tool to use when patients cannot be treated with intravenous tPA (because too much time has elapsed from the patient's ‘last seen normal’) or mechanical thrombectomy (because the composition of the clot would make it difficult or dangerous to remove).

### Example 1.1. Device Components

FIGS. 1, 2A-2C and 3A1-3B2 provide illustrations of a localized intra-arterial drug delivery device 10. The key functional components of the device are the lumen 12, which allows immediate restoration of blood flow, the active agent dispenser 14, which delivers the clot dissolving drug, and the irrigation channel 18, which removes clot pieces and drug out of the vessel. The rest of the device's components support these features.

In this Example, lumen 12 extends the length of the device. Additionally, lumen 12 immediately restores blood flow upon device insertion through the clot and bushing removal. Moreover, lumen 12 allows for some degree of blood flow during the entirety of drug delivery. Additionally, lumen 12 serves to introduce the optional expandable structure 16, if the clinician elects to remove any remaining fragments.

A bushing (not shown) allows the lumen 12 to also function as the lumen for the guidewire. These bushings keep the guidewire centered within the lumen 12, despite their substantially larger diameter.

Active agent dispenser 14 in this example is a helical, permeable, and hollow fiber that allows a thrombolytic agent to be delivered into direct contact with a blood clot. The helical shape provides high surface area for drug delivery and allows the active agent dispenser to remain in contact with the clot for direct contact with the thrombolytic agent. The active agent dispenser is initially wound onto the device exterior in such a way that it can expand outward radially to follow the surface of the dissolving clot. As illustrated in FIGS. 3A1-3B2, this is achieved with the help of a nitinol wire 23 that is attached to the active agent dispenser. Another possible use of the active agent dispenser 14 is to diffuse saline through it to facilitate filtration and reabsorption through the irrigation channel 18. The saline will be used to assist in the aspiration at the proximal part of the device to push the drug into the inlets of the device.

Expandable structure 16, which represents a balloon in this Example, is made by heated pressurization to fit a device-specific mold. Expandable structure 16 keeps a thrombolytic agent localized. This allows for higher drug concentration at the site of the clot than if the drug were administered systemically. This may also permit different potentially more potent drugs to be used than if the drug is allowed to travel systemically throughout the body. The expandable structure 16 can also be used to aid in centering the guide wire 17 before penetrating the clot. This ensures the device is implemented in a more central location of the clot, rather than towards the outer edges of the clot.

Irrigation channel 18 draws the drug out of the blood vessel after the clot has been sufficiently dissolved, preventing it from mixing with the rest of the circulatory system. Irrigation channel 18 interfaces with an external device for providing suction. The suction generating device may also monitor the volume of drug removed. A protective sheath (not shown) protects the device while it is being inserted into place.

As illustrated in FIGS. 1 and 2A-2B, inflating channel 20 serves two functions that are determined by the location of the valve stopper 24. Inflating channel 20 allows a drug to reach the active agent dispenser 14 and for fluid to inflate the expandable structure 16. Inflating channel 20 may interface with an external device for introducing pressurized drug or saline.

As illustrated in more detail in FIGS. 2A-2C, bypass lumen 22 has both ends connected to inflating channel 20. This allows for fluid to bypass the connection between active agent dispenser 14 and inflating channel 20 so that fluid can inflate the expandable structure 16.

Valve stopper 24 controls where fluids may advance in the inflating channel 20 and allows a single lumen to supply both the expandable structure 16 and active agent dispenser 14, thereby reducing the overall device diameter. In its deployment position, valve stopper 24 blocks both the bypass lumen 22 and entry site of the active agent dispenser 14 (FIGS. 3A1-3A2). This prevents the expandable structure from inflating and drug/saline from entering the fiber. In its inflation position, valve stopper 24 allows saline to enter bypass lumen 22 and reach the expandable structure 16 while still blocking the entry site of active agent dispenser 14 (FIGS. 3B1-3B2). In its dispensing position, valve stopper 24 blocks the distal end of the bypass lumen 22, thus keeping the expandable structure 16 inflated (FIG. 3C). In this position, the valve stopper 24 has advanced past the connection between the active agent dispenser 14 and the inflating channel 20, which allows drug to enter the active agent dispenser 14 and be delivered to the clot. Valve stopper 24 may be actuated by a stiff wire that extends proximally to the exterior of the device.

### Example 1.2. Device Operation

As illustrated in FIGS. 4A-4H, the device described in Example 1.1 can be operated as follows. First, a guide wire is inserted so that it nearly touches the blood clot (FIG. 4A). Next, catheter device 10 is advanced along the guidewire and stops before penetrating the clot (FIG. 4B). Expandable structure 16 is then inflated to center the guide wire and the device 10 prior to penetration of the clot (FIG. 4C). The guide wire is then pushed through the clot while the expandable structure is inflated to ensure the penetration is in a more central location of the clot (FIG. 4C). Next, the expandable structure 16 is deflated to allow the device to puncture through the center of the clot (FIG. 4D).

After the device 10 punctures through the clot, the valve stopper 24 is advanced to its inflating position (FIG. 4E). This allows fluid (which could be drug or saline) to enter the bypass lumen 22 and inflate the expandable structure 16. Inflating fluid is pumped by external means until the expandable structure 16 is sufficiently inflated. The guidewire 17, bushings, and protective sheath are removed. Once the bushings are removed, blood is free to flow through the lumen 12 to provide some degree of immediate recanalization.

Thereafter, valve stopper 24 advances to its dispensing position (FIG. 4F). In this position, it blocks the distal end of the bypass lumen 22, which traps fluid inside of the expandable structure 16, keeping it inflated. This is the only position of the valve stopper 24 that allows drug to enter the active agent dispenser 14. Once the valve stopper reaches this position, drug begins to diffuse out of the active agent dispenser 14 and dissolves the clot (FIGS. 4F-4G). The drug is pumped into the fiber by an external pump that monitors the total volume delivered. As the clot is gradually dissolved, the active agent dispenser 14 is permitted to unfurl slightly, causing it to expand outwards radially to maintain close proximity to the surface of the clot.

Once the clot has been sufficiently dissolved, a mixture of drug and blood is drawn out of the artery and back into the device through the irrigation channel 18. This suction is provided by an external device and can monitor the total volume extracted to ensure most or all of the delivered drug is recaptured (FIG. 4G).

If a clinician elects to use an expanding net, it is now deployed by extending it from the proximal end of the lumen. The net exits the distal end of this lumen and self-expands. Finally, valve stopper 24 is moved back to the inflating/deflating position, in which the bypass lumen is not blocked on either end. This allows the expandable structure to deflate (FIG. 4H). Once the expandable structure is deflated, the device can be removed from the patient.

The in vitro testing of a three-dimensional printed model of device 10 demonstrated successful inflation of the expandable structure in the blood clot analog, successful dispersion of the thrombolytic agent analog into the blood clot analog using the active agent dispenser, and successful deflation of the expandable structure for removal of the device after the thrombolytic agent has been removed via suction. Finally, in vitro testing of nitinol wire in the active agent dispenser showed that the nitinol is able to adjust its shape due to heat while enclosed in the active agent dispenser.

### Example 1.3. Summary

The intra-arterial localized drug delivery device in this Example was developed at least in part to treat acute ischemic stroke (AIS), especially in view of the shortcomings of the current methods of AIS treatment, such as intravenous tPA and mechanical thrombectomy (e.g., clot fragmentation, device embolization, and/or inadequate blood reperfusion times). This device immediately provides some restoration of blood flow upon insertion through the clot, which is crucial to save oxygen-starved tissue from dying. As a result, the device can potentially remain in the patient's artery for extended times as needed to sufficiently dissolve the clot. The use of local delivery coupled with the distal expandable structure allow the drug concentration at the site of the clot to be significantly higher than would be typical for more conventional systemic delivery. Additionally, as the drug is not released to the rest of the circulatory system, other drugs may be acceptable for use, such as those not approved for systemic use.

Moreover, device 10 prevents both device embolization and clot fragmentation, reducing the chance of emboli traveling further into cerebral vasculature. Additionally, the multi-channel nature of the device provides a significant advantage over other devices concerning blood reperfusion time. When the device is deployed, there is an almost-instantaneous flow of blood through an occluded vessel.

In summary, device 10 was developed at least in part to resolve pressing issues among currently used stroke clot devices. Garnering inspiration from the shortcomings of commercialized mechanical thrombectomy devices such as clot fragmentation, device embolization, and sometimes-inadequate blood reperfusion times, device 10 utilizes a novel mechanism of clot removal to address these shortcomings.

### Example 2. Alternative Design and Use of Active Agent Delivery Devices

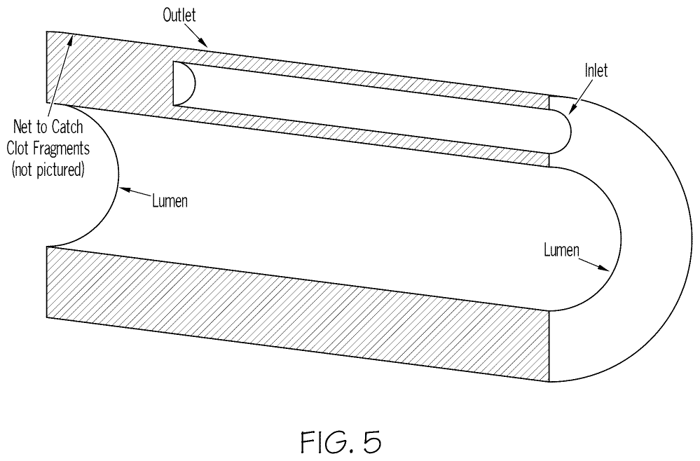

As illustrated in FIGS. 5 and 6A-6C, this Example provides an alternative design and use of an active agent delivery device. In this Example, the balloon is replaced with a net as the expandable structure. The expandable net can catch clot fragments and prevent further downstream clotting. The net can be mechanically controlled.

A need for an inflating channel to fill a ballon is no longer necessary for the device in this Example. Moreover, the device removes the ability to limit downstream flow of an active agent (e.g., a thrombolytic agent), but does maintain the ability of the device to immediately restore partial blood flow to the affected area.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. An active agent delivery device operable for insertion into a blood vessel, wherein the active agent delivery device comprises:
a proximal end, a distal end, and an exterior surface between the proximal end and the distal end;
a lumen extending from the proximal end to the distal end beneath the exterior surface, wherein the lumen is operable to facilitate blood flow through the blood vessel;
an active agent dispenser associated with the exterior surface, wherein the active agent dispenser is operable to release at least one active agent into the blood vessel;
an irrigation channel operable to remove materials from the blood vessel; and
an expandable structure at or near the distal end, wherein the expandable structure is operable to expand downstream the blood vessel region containing the active agent delivery device and thereby localize the released active agent proximal to the blood vessel region containing the active agent delivery device.

2. The active agent delivery device of claim 1, wherein the active agent dispenser is in the form of a helical membrane wound around the exterior surface.

3. The active agent delivery device of claim 1, wherein the active agent dispenser is operable to expand outward radially to follow a surface of the blood vessel by switching from a tilted position to an un-tilted position.

4. The active agent delivery device of claim 1, wherein the irrigation channel extends from the proximal end to the distal end in parallel with the lumen, and wherein the irrigation channel is operable to interface with a suction device for the removal of materials from the blood vessel.

5. The active agent delivery device of claim 1, wherein the expandable structure is selected from the group consisting of a balloon, a net, or combinations thereof.

6. The active agent delivery device of claim 1, wherein the expandable structure comprises a balloon.

7. The active agent delivery device of claim 1, further comprising an inflating channel associated with the expandable structure, wherein the inflating channel is operable to expand the expandable structure through the flow of air or fluids into the expandable structure, and wherein the inflating channel is operable to interface with a fluid or air delivery device for expansion of the expandable structure.

8. The active agent delivery device of claim 1, further comprising an active agent delivery channel associated with the active agent dispenser, wherein the active agent delivery channel is operable to deliver one or more active agents to the active agent dispenser, and wherein the active agent delivery channel is operable to interface with an active agent delivery pump for active agent delivery to active agent dispenser.

9. The active agent delivery device of claim 1, further comprising:
an inflating channel associated with the expandable structure, wherein the inflating channel is operable to expand the expandable structure through the flow of air or fluids into the expandable structure;
an active agent delivery channel associated with the active agent dispenser and the inflating channel, wherein the active agent delivery channel is in air or fluid communication with the active agent dispenser and operable to deliver one or more active agents to the active agent dispenser; and
a valve stopper associated with the inflating channel and the active agent delivery channel, wherein the valve stopper is operable to cease air and fluid flow to the inflating channel and the active agent delivery channel, direct air and fluid flow to the inflating channel but not the active agent delivery channel, direct air and fluid flow to the active agent delivery channel but not the inflating channel, or direct air and fluid flow to the active agent delivery channel and the inflating channel.

10. A method of delivering an active agent into a blood vessel of a subject, said method comprising:
inserting an active agent delivery device into the blood vessel, wherein the active agent delivery device comprises:
a proximal end, a distal end, and an exterior surface between the proximal end and the distal end,
a lumen extending from the proximal end to the distal end beneath the exterior surface,
an active agent dispenser associated with the exterior surface,
an irrigation channel, and
an expandable structure at or near the distal end;
expanding the expandable structure downstream the blood vessel region containing the active agent delivery device; and
delivering at least one active agent from the active agent dispenser into the blood vessel.

11. The method of claim 10, wherein the lumen facilitates blood flow through the blood vessel, the irrigation channel removes materials from the blood vessel, and the expanded expandable structure localizes the released active agent proximal to the blood vessel region containing the active agent delivery device.

12. The method of claim 10, wherein the released active agent dissolves clots in the blood vessel.

13. The method of claim 10, further comprising a step of coupling the irrigation channel with a suction device, wherein the suction device facilitates the removal of materials from the blood vessel.

14. The method of claim 10, wherein the active agent delivery device further comprises an inflating channel associated with the expandable structure, and wherein the expanding comprises flowing air or fluids into the inflating channel to expand the expandable structure.

15. The method of claim 14, further comprising a step of interfacing the inflating channel with a fluid or air delivery device for expansion of the expandable structure.

16. The method of claim 10, wherein the active agent delivery device further comprises an active agent delivery channel associated with the active agent dispenser, wherein the active agent delivery comprises delivering one or more active agents to the active agent dispenser through the active agent delivery channel.

17. The method of claim 16, further comprising a step of interfacing the active agent delivery channel with an active agent delivery pump for active agent delivery to the active agent dispenser.

18. The method of claim 10, wherein the active agent delivery device further comprises: an inflating channel associated with the expandable structure, an active agent delivery channel associated with the active agent dispenser and the inflating channel, and a valve stopper associated with the inflating channel and the active agent delivery channel, and wherein the method further comprises:
positioning the valve stopper in a first position to direct air or fluid flow to the inflating channel but not the active agent delivery channel, wherein the air or fluid flow expands the expandable structure downstream the blood vessel region containing the active agent delivery device; and
positioning the valve stopper in a second position to direct air or fluid flow to the active agent delivery channel but not the inflating channel, wherein the air or fluid flow delivers at least one active agent from the active agent dispenser into the blood vessel while the valve stopper maintains the expandable structure in an expanded position.

19. The method of claim 10, wherein the active agent is selected from the group consisting of a thrombolytic agent, a clot dissolving drug, and anti-tumor drug, plasmin, or combinations thereof.

20. The method of claim 10, wherein the blood vessel is selected from the group consisting of an artery, a vein, a blocked blood vessel, or combinations thereof.
